(19) United States
(12) Patent Application Publication
(10) Pub. No.: US 2005/0273367 A1
(43) Pub. Date: Dec. 8, 2005

(54) Secure health information connectivity system

(75) Inventors: Michael Nourie, San Carlos, CA (US); Matthew K. Hudes, Los Gatos, CA (US); Roy Benjamin, Sunnyvale, CA (US); David A. Chen, Half Moon Bay, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Accelere, Inc., Los Gatos, CA (US)

(21) Appl. No.: 11/129,652

(22) Filed: May 12, 2005

Related U.S. Application Data
(60) Provisional application No. 60/571,675, filed on May 14, 2004.

(51) Int. Cl. G06F 17/60; G21K 1/12; A61B 6/00; G01N 23/00

(52) U.S. Cl. 705/3

(57) ABSTRACT

A method is disclosed. The method includes receiving site specific patient information including a plurality of site specific data sets for a plurality of patients from different sites at a server computer, where the site specific patient information was previously converted from a first data format to a second data format. The site specific patient information including the site specific data sets is converted into standard patient information including standard data sets at the server computer.

### CROSS-REFERENCES TO RELATED APPLICATIONS

This application is a non-provisional of and claims the benefit of U.S. Provisional Application No. 60/571,675, filed on May 14, 2004, which is herein incorporated by reference in its entirety for all purposes.

This application is also related to U.S. patent application Ser. No. ______ (attorney docket no. 025894-000110US) entitled “Patient Recruitment Method And System”, which is being filed on the same day as the present application and which is herein incorporated by reference in its entirety for all purposes.

### BACKGROUND OF THE INVENTION

Clinical trials are used to test the efficacy and safety of new drugs. In clinical trials, patients are recruited to form test groups. The patients in the test groups need to have particular characteristics which are relevant to the drugs being evaluated. For example, if a drug that is being tested is for treating prostate cancer, only males within a predetermined age range would be suitable candidates for the drug study.

Suitable candidates for clinical trials and other studies can be found in any number of ways. In one conventional method, using some predetermined criteria, a study coordinator helps a study investigator (e.g., a doctor) comb through archives of paper or electronic records at a healthcare facility such as a hospital to find suitable candidates. Patients that match the predetermined criteria are identified as being suitable study candidates. Their consent to participate in the study is then obtained. In another conventional method, an advertisement for study participants is taken out in an advertising medium such as a newspaper. The advertisement mentions the criteria for the study participants and the study coordinator's contact information. After potential candidates see or hear the advertisement, they contact the study coordinator. The study coordinator then determines if the candidates are suitable for the study. If the potential candidates are suitable, their consent to participate in the study is obtained by the coordinator.

Conventional methods for recruiting patients for clinical studies are effective in some instances. However, there are a number of problems associated with conventional patient recruitment methods. For example, the recruitment process is expensive and time-consuming. Patient recruitment costs more and consumes more time than any other aspect of clinical trials. In fact, more than 80% of clinical trials suffer from recruitment delays. Patient and site (investigator) enrollment accounts for 41% of the time spent on clinical research. The delays associated with patient recruitment inevitably delays the introduction of new drugs and therapies to the public.

Drug manufacturers, in particular, are sensitive to the delays associated with patient recruitment and enrollment. Drug manufacturers invest a significant amount of money researching and developing new drugs and getting government approval to sell the new drugs. Because of the large capital investment associated with developing new drugs and bringing them to market, drug manufacturers want to reduce the amount of time needed to bring the drugs and therapies to market. The drug manufacturers want to see a return on their initial investments as soon as practical.

The delays associated with conventional recruitment and screening methods also preclude many potential candidates from participating in studies and limit the types of studies that can be conducted. For instance, a study may require the collection of time-sensitive patient data. An exemplary study may require measuring a patients' blood pressure during an asthma attack or shortly thereafter. Assuming that this type of study could even be conducted using traditional patient recruitment processes, it would be difficult to obtain the patients' consent and then obtain their blood pressure data in a timely manner using the slow conventional recruitment processes. This is because suitable candidates need to be identified, and the patients' consent and blood pressure data needs to be obtained in a short time period. Data needs to be obtained in a short period for it to be relevant to the study.

Screening patients for a study and getting patients to consent to participate in a study can be challenging, especially when complex inclusion/exclusion criteria are applied to the screen patients. As noted above, it is also difficult when screening for in-patient, acute indications where timing is critical (e.g., unscheduled surgery, emergency visits, cardiovascular and respiratory episodes). There is also an increased concern for patient privacy, and data collection errors can also add complexity to the screening process.

It would be desirable to provide for a recruitment and screening solution that finds patients for studies, such as clinical trials and observational studies, based on active health care information, including patient medical record information currently housed within registration, laboratory, and pharmacy systems. It would also be desirable to provide for the use of real-time information to build a clinical record, and to perform fact-based patient population forecasting and recruitment in a HIPAA-compliant and secure manner. (HIPAA, or the Health Insurance Portability and Accountability Act, was passed by Congress in 1996 to set a national standard for electronic transfers of health data.)

Embodiments of the invention address these and other embodiments of the invention individually and collectively.

### SUMMARY OF THE INVENTION

Embodiments of the invention are directed to methods and systems for recruiting and screening patients for studies such as clinical trials or observational studies.

One embodiment of the invention is directed to a method comprising: receiving site specific patient information including a plurality of site specific data sets for a plurality of patients from different sites at a server computer, wherein the site specific patient information was previously converted from a first data format to a second data format; and converting the site specific patient information including the site specific data sets into standard patient information including standard data sets at the server computer.

Another embodiment of the invention is a system comprising: a data connect element adapted to convert site specific patient information for a plurality of patients from a first format to a second format; and a server computer adapted to convert the site specific patient information to standard patient information.

Another embodiment of the invention is directed to a method of selecting a candidate for a study, comprising: receiving a plurality of patient data sets at a data store located within an internal network, wherein each data set comprises at least one site specific term and an associated site specific patient value; processing each of the received data sets as needed to transform the data sets into private data sets, and further, wherein the processed and private data sets are compatible with transport over an external network; communicating the processed data sets over the external network to an external data store; processing the communicated data sets as needed to transform each of the data sets into a standard data set including a canonical term and an associated canonical value; comparing the transformed communicated data sets to a study scenario comprising a plurality of study parameters including the canonical data term and at least the associated canonical data value; and processing the compared data sets to select a candidate data set for a study.

These and other embodiments of the invention are described in further detail below.

### BRIEF DESCRIPTION OF THE DRAWINGS

FIG. 1 shows a flowchart showing a general method according to an embodiment of the invention.

FIG. 2 shows a block diagram of a system according to an embodiment of the invention.

FIG. 3(a) shows a block diagram of an exemplary data connect element.

FIG. 3(b) shows a block diagram of an exemplary data central server.

FIG. 4(a) shows a flowchart showing an exemplary process.

FIG. 4(b) shows a flowchart showing an exemplary workflow process.

FIGS. 5-14 show screenshots of graphical user interfaces that can be used in a method according to an embodiment of the invention.

### DETAILED DESCRIPTION

Embodiments of the invention are directed to recruitment solutions that find patients for clinical or observational trials based on healthcare information such as current healthcare information. The healthcare information includes patient medical record information currently housed within registration, laboratory, and pharmacy systems. In embodiments of the invention, real-time information is used to build a clinical record, and to perform patient population forecasting and recruitment in a HIPAA-compliant and secure manner. Embodiments of the invention also use networks that allow investigators, study coordinators, site coordinators, etc. to connect to data sources at healthcare institutions to access real-time patient information and conduct workflow processes in a HIPAA-compliant manner. Using embodiments of the invention, pharmaceutical and biotechnology research sponsors can also obtain patient information in a HIPAA-compliant manner in real time.

The technology-based recruitment and data management solutions according to embodiments of the invention overcome traditional clinical research barriers. Investigators can focus on patient care and/or patient research, rather than combing through large archives of paper or electronic patient files searching for suitable study candidates. Embodiments of the invention allow investigators to automate the patient recruitment process across multiple sites with different data standards using a uniform scenario.

Using embodiments of the invention, trial planning, site/investigator selection, patient recruitment, clinical data collection and status reporting are accelerated. In some embodiments, study investigators or coordinators can be paged or otherwise notified when potential candidate patients are found for the study. They can also access critical patient information and drive the patient workflow process from their wireless devices. Embodiments of the invention shorten the time to market for new drugs and facilitate the tracking of quality metrics in healthcare settings.

Also, embodiments of the invention can be used to identify a potential participant base for a clinical trial before filing a new drug application with the FDA (food and drug administration) or other regulatory organization. This provides for better managed and shorter clinical trials. For example, by identifying a potential participant base for a clinical trial in advance, an investigator can determine how many site coordinators will be needed to coordinate the study. In another example, if the system identifies a small number of currently available patients, the investigator can make decisions about whether to extend the time period for patient recruitment or adjust the study requirements to include fewer patients.

A number of entities can benefit using embodiments of the invention. Those entities include life science companies, healthcare provider organizations, clinical research organizations, drug companies, universities, hospitals, and patients who need new therapies.

Any suitable study may be performed in embodiments of the invention. For instance, the study may be an observational study or may be clinical trial. The study may be used to test for the efficacy and/or any potential problems (e.g., toxicity) associated with a new drug, a new medical procedure, a new therapy, or a new medical device. The study may be a study that is conducted before approval is given for a drug, medical device or the like, or may be study that is conducted after approval is given for a drug, medical device or the like. Post approval studies can be conducted to verify the efficacy or safety of the drug, medical device, or the like.

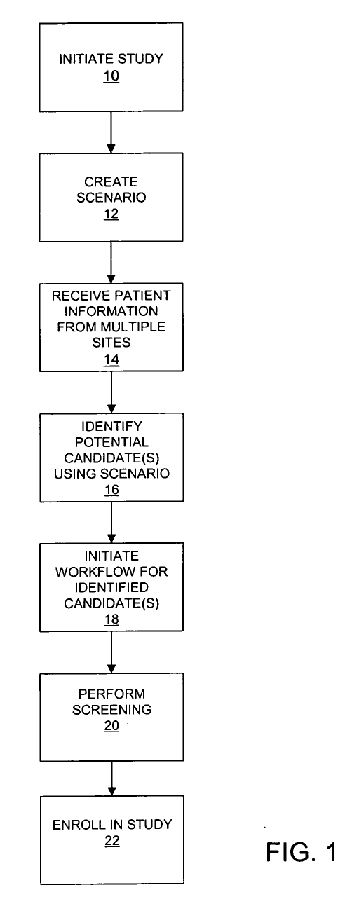

FIG. 1 shows a flowchart illustrating a general method according to an embodiment of the invention. In other embodiments of the invention, steps may be omitted or added to the steps shown in FIG. 1.

As shown in FIG. 1, a study is initiated (step 10). The study is typically initiated by an investigator such as a doctor, a clinical researcher, a university professor, or a research scientist. The investigator may work with a study coordinator and/or one or more site coordinators, who may help the investigator screen a patient population for potential study candidates and/or assist with the workflow process for getting those study candidates into the study. For example, the study coordinator and/or the site coordinator may help obtain the consent of potential patients to participate in the study.

“Site coordinators” are typically individuals located at the different sites that provide the patient information to be screened. For example, different hospitals may have respectively different site coordinators to coordinate the patient workflow processes at those sites. They may obtain patient consent, get patient samples, etc. A typical site coordinator may be a nurse or a medical technician. A “study coordinator” may be an individual who coordinates a study for an investigator. The investigator may also be a study coordinator in some embodiments.

After the study is initiated, a scenario is created (step 12). The scenario can be created by the investigator using a client computer. An investigator may use a menu driven software program on the client computer or at a remote site to create it. Once created, the scenario may be uploaded to a data central server where the scenario will be run against patient information from different sites.

The scenario defines the desired patient medical characteristics for the study. It is typically comprised of one or more scenario parameters. Each scenario parameter may have a scenario term (which may be the same as or correspond to a study specific term or a standard term), which may have a value or range of scenario values associated with it. For example, a typical scenario for a study to monitor the sodium and potassium levels in older smoking females over time might be:
Critical Potassium (K<2.5 or K>6)
Critical Sodium (Na<120 or Na>160)
Female Smoker (yes or no)
Age (>35)
   In this example, scenario terms might be “critical potassium”, “critical sodium”, “female smoker”, and “age”. In this and other scenarios, one or more scenario values may be associated with the one or more scenario terms. The one or more scenario values associated with the terms in the above scenario would respectively be “(K<2.5 or K>6)”, “(Na<120 or Na>160)”, “(yes or no)”, and “(>35)”. As illustrated in this example, the one or more scenario values associated with a scenario term may be binary in nature (e.g., yes or no) or may form a range values (e.g., >35). The scenario values may also have units associated with them. For example, the units for term “critical potassium” might be in milliequivalents per liter.

A study-specific workflow may also be configured when the study is initiated. Specifically, a patient workflow process is configured for each study to govern the actions of the study coordinators during the screening process, and the patient data entry process. The workflow process may be conducted so that it satisfies the requirements of HIPAA and/or privacy rules dictated by some other law or organization. When complying with the requirements of HIPAA, for example, certain different levels of patient information may be accessible to different individuals. For instance, doctors may be able to see more patient information about their patients than study investigators.

The steps for screening may differ in the different studies and different requirements and/or forms may be used in the different studies. In other embodiments, the screening steps and forms may be the same for different studies. As explained in further detail below, once a patient is enrolled, a series of patient “visits” can be conducted by a study or site coordinator over a period of time. Data entry “alerts” and patient “drop-out” actions can be driven from this workflow.

Embodiments of the invention also allow an investigator or other user to configure data “outlier” alerts for specific data elements. For example, a system according to an embodiment of the invention can provide the option to specify outlier parameters (e.g., minimum population, variations in terms of standard deviations, etc). Also, the system can provide the investigator with the ability to specify the source of the patient information that is used for the study. For example, the desired population information may include one type of data value over a series of data entry instances for one patient (e.g., potassium readings for one patient over several visits), or one type of data value over a series of data entry instances for a series of patients (e.g., many potassium readings for many patients over single visits).

After the scenario is created and the workflow is defined, patient information is then received from multiple sites (step 14). (In other embodiments, patient information can be received first, and the scenario and the workflow can be defined at a later time.) The patient information that is received from the different sites may include various site specific data sets associated with different patients. The patients may be at or affiliated with those sites. Each patient may have one or more site specific data sets associated with that patient. For each patient, each site specific data set may include a site specific term (e.g., body temperature) and a site specific patient value (e.g., 98) associated that site specific term. Optional units may be associated with the site specific patient value (e.g., Fahrenheit). Some values, such as binary values (e.g., yes/no) may not have units associated with them.

The different sites from which patient information is obtained may include different healthcare-related entities. Examples of healthcare-related sites include hospitals, pharmacies, doctor's offices, medical clinics, heath maintenance organizations, test laboratories, etc. The sites may also include non-healthcare related sites such as patients' homes. In some cases, a patient initiates a data transfer. For instance, the results of a home EKG test, blood pressure test, blood test or urine test may be transmitted over the phone or wireless network to a central server or a healthcare facility. Other sites may be entities such as insurance companies which may house patient information.

The different sites may be at different locations. However, in other embodiments, different sites may be at the same location. For example, a pharmacy and an emergency room in a hospital may be considered different sites, yet may be at the same location.

After the patient information including the site specific data sets is received at a data central server from the different sites, potential candidates for the study are then identified using the created scenario (step 16). In some cases, the scenario may be present at the different sites and potential candidates may be identified at those different sites instead of at a data central server. If the created scenario is uploaded to the data central server, the data central server can run the scenario against patient information received from the different sites to find patients that meet the criteria of the scenario. The patient information received at the data central server may be temporarily or permanently stored in a data store at the data central server. The data central server may be external to and remotely located with respect to the sites.

As explained in detail below, the patient information that is received from the different sites is converted into a common format so that the scenario can be used to screen the patient information. More specifically, the site specific data sets can be transformed, as needed, into “standard” data sets. A site specific data set includes terms (e.g., “potassium level”), values (“2.0”), and units (e.g., “milliequivalents per liter”) that are specifically used by a particular site. Standard data sets include terms, values, and units that are standardized to a predetermined standard. The standard data sets may be subsequently transformed into study specific data sets. Study specific data sets include terms, values, and units that are specific to the study being conducted. The scenarios according to embodiments of the invention may also use terms, values, and units that are specific to the study being conducted, or may use other terms, values, and units.

Each site specific data set can thus include a site specific term that is transformed into a standardized term, which may also be a canonical term with respect to the site specific term. A canonical term may be a different way to express another term and a canonical value may be a different way to express another value. In some embodiments, a canonical value may be derived from or may relate to a patient specific value. For instance, a site specific patient value can be transformed into a standardized patient value, which may also be a canonical value with respect to the site specific patient value. The site specific patient specific value may be transformed in any suitable way to produce a canonical value. For example, the site specific patient value may to multiplied or divided by a conversion factor to produce a canonical value such as a standardized patient value or a study specific patient value.

The scenario can be run against patient information from any suitable number or combination of sites using the data central server or another other computational apparatus. As will be shown below, the system according to embodiments of the invention can produce a comprehensive report outlining the number of potential candidates by acceptance criteria, physician, and/or site. Report parameters in the comprehensive report may also be a form a data filtering. The system also provides for the ability to save pre-configured scenario views (i.e., report parameters) in a user folder for routine on-request reporting. This saves the investigator or other users from repeatedly re-selecting parameters.

In embodiments of the invention, the investigator has the ability to run the scenario against live site data to drive a report. This functionality is desirable for ad-hoc patient listings based on pre-defined recruitment scenarios to drive recruitment activities. For example, a site may want to run a specific scenario and generate a current patient listing before each set of recruitment rounds to determine the “most-likely” trial candidates by ICU (intensive care unit) based on blood gas results.

A workflow is then initiated with respect to the identified candidates (step 18). The workflow process includes verifying that the identified candidates are in fact suitable for the study and obtaining the consent of any verified and identified candidates. These steps may also form a screening process (step 20).

The workflow process may be conducted in a manner that complies with a predetermined set of rules relating to the privacy of patient information. For example, the workflow process may comply with HIPAA or may comply with patient information disclosure rules created by a governance committee. An example of a governance committee is a CHR (committee on human research). A typical governance committee may review studies involving human subjects, including research methods, recruitment techniques, study procedures, consent forms and all other appropriate forms, documents and survey instruments. Although HIPAA is described in detail in this application, it is understood that the workflow process may be designed to comply with HIPAA, or any other set of rules that relate to patient privacy.

Preferred embodiments of the invention “electronically govern” the workflow process so that it proceeds in a secure, efficient, and HIPAA compliant manner. Some of the workflow parameters may be dictated by HIPAA (or other set of rules) and some may be dictated by the particular study. Embodiments of the invention allow the investigator and study coordinator to see only what is necessary while also driving the process of getting the consent and participation of the identified patients. For example, investigators are often authorized to view specific data for specific patients for a set time frame (e.g., 3 months). Embodiments of the invention ensure compliance with these time frames through workflow driven data entry screens, and back end reporting tools. Where necessary, some data is “de-identified”. That is, patient information is obtained and information that is not used is deleted from the system and is no longer identifiable by the investigator or coordinators. This is done to protect patient privacy. Further details regarding methods according to embodiments of invention are provided below.

After the patients are selected for the study, the patients are then enrolled in the study (step 22). The study is thereafter conducted. However, as will be explained below, embodiments of the invention also provide some tools such as assisted data entry screens to help the investigator accurately enter data and conduct the study.

Steps 12, 14, and 16 can be considered general steps in a candidate “recruitment” process. In embodiments of the invention, the recruitment process can take place in substantially “real time” (e.g., identifying a candidate patient in less than 10, 5, or 1 minute after patient information is first obtained at a site). Steps 18, 20, and 22 can be considered general steps in a workflow process according to an embodiment of the invention.

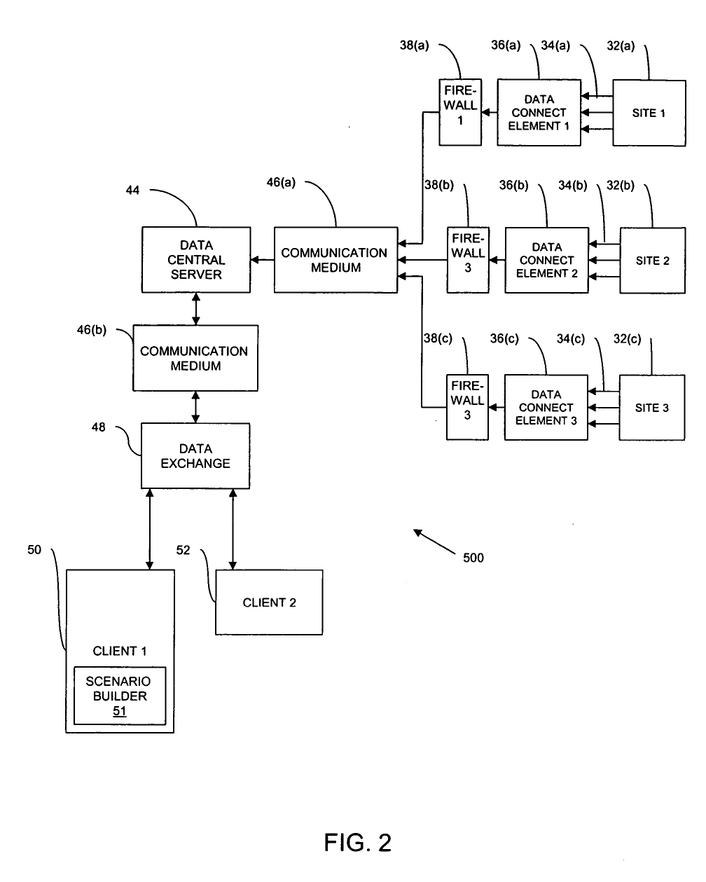

FIG. 2 shows a system 500 according to an embodiment of the invention. The system includes a number of different sites 32(a)-32(c). The different sites 32(a)-32(c) may be healthcare facilities such as hospitals, doctor's offices, medical clinics, patients' homes, etc.

Data connect elements 36(a)-36(c) may be at the respective sites 32(a)-32(c). Firewalls 38(a)-38(c) may be respectively associated with the data connect elements 36(a)-36(c). The data connect elements 36(a)-36(c) may be servers located at the respective sites 32(a)-32(c). Further details regarding exemplary data connect elements are provided below.

The data connect elements 36(a)-36(c) can be in communication with multiple data feeds 34(a)-34(c). In some embodiments, the data feeds 34(a)-34(c) may be HL7 data feeds. HL7 stands for “Health Level 7”. HL7 is a standard used for storing and interchanging clinical and administrative data. The HL7 data feeds may carry patient information comprising, for example, patient medical history and demographics, encounter and visit histories, admit/discharge/transfer and patient tracking information, scheduling and referrals, orders and results (measurements, observations, impressions, reports), pharmacy and diet information, and census information. In other embodiments, the data feeds 34(a)-34(c) could carry messages of another type (e.g., SMS messages).

The data feeds 34(a)-34(c) may be connected to different data sources. The data sources and the data connect elements 36(a)-36(c) may be respectively connected to each other via an internal network such as an Ethernet. Such data sources may include pharmacies, labs, doctors' offices, etc.

The data connect elements 36(a)-36(c) communicate with a data central server 44 through a communication medium 46(a). The data central server 44 may include software components for performing a number of functions including decrypting patient information, converting patient information into a standard format, applying a scenario to the standardized patient information, converting selected patient information into study specific information, and sending study specific information to one or more client computers operated by an investigator, study coordinator, or site coordinator. In some embodiments, the scenario (in a standardized or site specific format) may reside in the data connect elements 36(a)-36(c) so that the data connect elements 36(a)-36(c) only send information filtered by the scenario to the server 44.

Any of the functions described above may be embodied as computer code on any suitable computer readable medium. For example, the server 44 may include a computer readable medium comprising code for receiving patient information for a plurality of patients from a plurality of sites at the server, and code for selecting at least one patient using the received patient information and using a scenario, where the at least one patient is a candidate for a study.

The communication medium 46(a) can include at least one network, which may include at least one of a wireless network, the Internet, the World Wide Web, a Local Area Network (LAN), a Wide Area Network (WAN), etc. The at least one network that allows for communication between the data connect elements 36(a)-36(c) and the data central server 44 may be an “external” network with respect to the sites 32(a)-32(c).

The data central server 44 may be in communication with a data exchange 48 though a communication medium 46(b) like the previously described communication medium 46(a). The data exchange 48 may also be comprised of one or more servers, and facilitates data exchange between the client computers 50, 52, and the data central server 44. Research sponsors such as pharmaceutical companies (not shown) may also be connected to the data exchange 48. Such research sponsors may want to monitor the progress of the studies that they are sponsoring.

A number of client computers 50, 52 may be in communication with the data exchange 48. The client computers 50, 52, may communicate with the data central server 44 using any suitable networking communication channels.

Any suitable client computer may be used in embodiments of the invention. The client computers 50, 52 may each comprise a microprocessor, and may also include suitable input and output devices (displays, keyboards, speakers, etc.) operatively coupled to the microprocessor. The client computers 50, 52 may be stationary computers or may preferably be wireless devices such as wireless phones, personal digital assistants (PDAs), personal e-mail devices such as Blackberry™ devices, laptop computers, tablet PCs, etc. The wireless devices may use any suitable wireless technology including 802.11 wireless LAN technology. Wireless devices are particularly preferable, since decisions regarding whether or not identified candidates are suitable for the intended study need to be made quickly. Wireless devices allow the investigators and the coordinators to be notified of potential candidates for the study at any time and at any place. Wireless tablet PCs are desirable, since they can be used to capture the signatures of consenting patients.

A scenario builder 51 may be present on the one or more clients 50, 52 in the system 500. The scenario builder 51 is software that allows an investigator to build a scenario for the desired study. The scenario defines terms which define a desired patient population (e.g., a male between 35 and 40 years hold, blood type B positive, body temperature between 100-102° F., etc.). The scenario builder 51 may be menu-driven software which allows an investigator to build a custom scenario for the desired study.

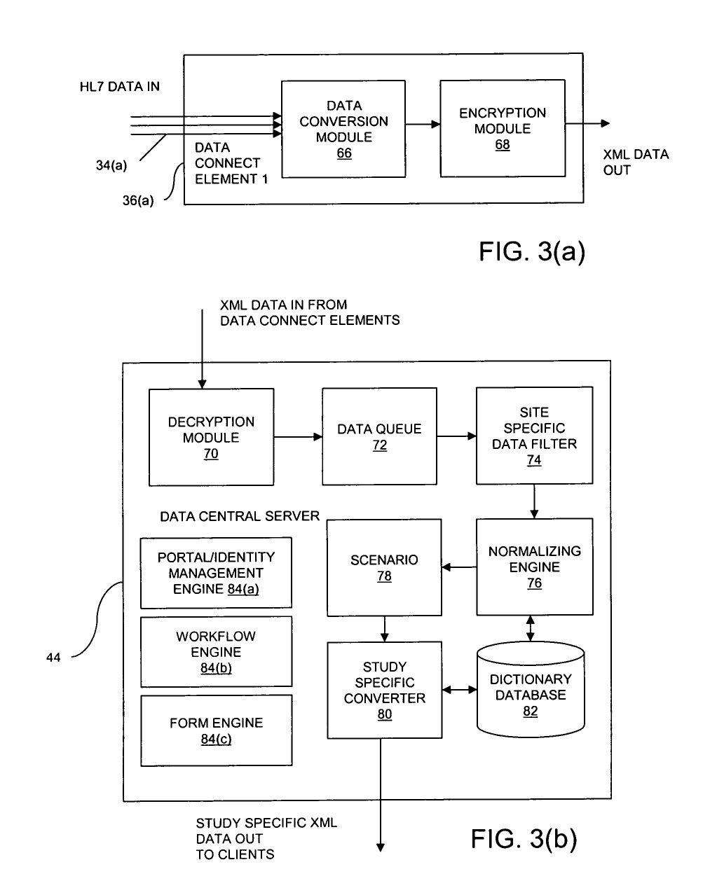

FIG. 3(a) shows a block diagram of one data connect element 36(a) according to an embodiment of the invention. The data connect element 36(a) includes a data conversion module 66 and an encryption module 68. As shown, there can be multiple data feeds 34(a) that input HL7 data to the data connect element 36(a). The data conversion module 66 converts the HL7 data into another data format, such as XML data. Although XML is described in detail herein, it is understood that other embodiments may use other markup languages such as HTML, SGML, etc. Then, an encryption module 68 encrypts the XML data before it is sent to the communication medium 46(a). Information that is received by the data connect element 36(a) may have been converted to HL7 data if the information was previously in a different format.

The data connect element 36(a) can be designed to forward the messages in a guaranteed manner as XML documents to data central server 44. The data connect element 36(a) can be deployed behind a health care provider's Internet firewall, where it will accept socket connections from trusted sources to receive HL7 messages. It can also make outbound socket connections to send XML documents. Outbound connections can use the SOAP (Simple Object Access Protocol) protocol.

The data connect element 36(a) can be configured to accept messages concurrently from multiple HL7 sources. Each HL7 source may send the same or different versions of HL7 messages. HL7 sources are defined in the data connect elements' static configuration, which can be defined in a local XML file. When the data connect element 36(a) is started, it reads the static configuration and start the services defined by it. The data connect element 36(a) can be configured so that it is modified at run-time and changes can be reflected immediately in the data connect elements' functions. The configurations can be saved as a new static configuration if desired.

The data connect element 36(a) can also support burst and “real-time” messaging. In burst messaging, the data connect element 36(a) sends available message data as a batch when a data or time threshold is passed. In real-time messaging, the data connect element 36(a) sends each HL7 message to the data central server 44 immediately upon receipt. Each HL7 message source can be configured to use one of these two messaging modes, i.e., the server may send messages from one HL7 source in batch mode, but use the real-time mode for messages from a different HL7 source. The data connect element 36(a) can also permanently or temporarily store HL7 messages locally (e.g., in an internal data store) until receipt of positive acknowledgement of XML document delivery. Temporary data storage is preferred since HL7 messages are continuously being received by the data connect element 36(a).

The data connect element 36(a) provides a high level of reliable and secure message delivery by using industry standards such as Sun Microsystem's Solaris operating system, Sun Microsystem's Java programming language, SOAP, XML, TCP/IP, and SSL.

The data connect element 36(a) may comprise a server that is a powerful computer or cluster of computers that behaves as a single computer which services the requests of one or more client computers. As used herein, a “server computer” can be a mainframe computer, a minicomputer, or a minicomputer cluster. For example, the data connect element 36(a) may be embodied by one or more Sun Fire V20 servers commercially available from Sun Microsystems.

Suitable characteristics of an exemplary data connect element are noted below. It is noted that such characteristics are exemplary and computing requirements and software tools may change over time and may be different in other embodiments.

| | |
|---|---|
| Hardware | 2 GHz processor (2) |
| | 2 GB memory |
| | 2 NIC |
| | RAID controller |
| | 300 gByte disk drive (2) |
| | CDROM |
| | 56 Kb Modem |
| | Uninterrupted Power Supply Pro 1400VA |
| | (remote access/control) |
| System Software | Solaris |
| Supporting Software | SUN WEB Server |
| | Java Application Server |
| | (Servlet/JSP Specifications 2.3/1.2) |
| | Sun ONE Java Application Server 7 |
| | Java J2SE 1.4.1_01 |
| | Java J2EE 1.3 |
| | Jakarta Log4J 1.2.8 |
| | SOAP v1.1 (Apache SOAP v2.3.1) |
| | Apache Xerces Java Parser v1.4.4 |
| | HAPI v0.3 (HL7 2.x parser) |
| | VNC |
| Environment | Power |
| | 19 inch rack mount space |
| | Dedicated phone line (2) |
| | LAN connection(s) to HL7 sources |
| | LAN connection (route to Internet) |

The data conversion module 66 translates HL7 messages into XML. Each HL7 message may include a site specific data set, which may include a site specific term and a site specific patient value. The data conversion module 66 is also responsible for putting the HL7-XML document in an output queue for transmission to the data central server 44.

FIG. 3(b) shows a block diagram of data central server 44. Data central server 44 may be comprised of one or more server computers. The data central server 44 may be a powerful computer or cluster of computers that behaves as a single computer which services the requests of one or more client computers. For instance, the data central server 44 may include one or more database servers coupled to one or more Web servers. For example, the data central server 44 may comprise a Sun Fire V240 server running Oracle database software.

Data central server 44 may include a number of components. Such components include, for example, a decryption module 70, a data queue 72, a site specific data filter 74, a normalizing engine 76, a scenario 78, a study specific converter 78, and a dictionary database 80. These components may be operationally coupled to each other. The data central server 44 may also include a data store (e.g., data queue 72) that may be considered an external to the sites from which the patient information originates. Patient information including patient data sets can be stored temporarily or permanently in the external data store.

The site specific data filter 74 filters the incoming patient information and patient data sets according to their originating site. As noted above, patient data sets arrive at the data central server 44 from different sites at different times and in different quantities. The site specific data filter 74 helps to prevent investigators from seeing information that they do not need to see. As noted above, patient information that investigators may see may be governed by a predetermined set of privacy rules (e.g., rules defined by HIPAA and/or a governance committee).

The normalization engine 76 changes site specific data sets into standard data sets. Site specific data sets may include site specific terms, site specific patient values, and site specific units. Site specific terms may be changed to standardized terms, and site specific units (if present) are changed to standardized units. Site specific patient values are also converted to standardized patient specific values.

The study specific converter 78 converts standardized data sets into study specific data sets. Study specific data sets may include study specific terms, study specific patient values, and study specific units. Standardized terms are changed to study specific terms and standard units are changed to study specific units. Standard patient values may also be converted to study specific patient values. A study specific converter 78 may or may not be needed in all embodiments. For example, if the investigator uses standard terms and standard units in a study, then a study specific converter is not needed.

The dictionary database 80 may include a mapping element that correlates the site specific terms and units, standardized terms and units, and study specific terms and units. As noted, site specific terms are terms that are used by the different sites which provide the patient information. Standardized terms are terms which are similar to terms used in the scenario. Study specific terms are terms which are used by the investigator. The dictionary database 80 may also store conversion factors for converting site specific patient values into standard values, and then into study specific values. The mapping between site specific terms and units, standardized terms and units, and study specific terms and units is described in further detail below.

Other software components 82 such as a portal identity management engine 84(a), a workflow engine 84(b), and a forms engine 84(c) may also be present in the data central server 44. Each of these components is described in further detail below.

The portal identity management engine 84(a) can manage access to the patient information. As noted above, in order to comply with HIPAA, it is desirable to provide investigators and coordinators with only the information that they need to conduct the study. Other information that would not be pertinent to the study may be excluded by the portal identity management engine 84(a). The portal identity management engine 84(a) may “de-identify” patients if they do not satisfy the scenario and/or may limit the amount of patient data sent to the investigator or coordinator. In a de-identification process, information about such patients may be completely removed from the system.

The workflow engine 84(b) manages the workflow processes for the patients. As noted above, during the workflow process, various messages may be sent to the coordinator and investigator to prompt them to take specific actions during the patient screening and enrollment processes. The workflow engine 84(b) may perform these functions.

The forms engine 84(c) may provide the appropriate forms for the coordinator and investigator at appropriate times during the various recruitment, screening, enrollment, and study processes. The forms engine 84(c) works in conjunction with the workflow engine 84(b) in some cases.

The engines 84(a), 84(b), 84(c), and any other software components or functions described in this application, may be implemented as software code to be executed by a processor using any suitable computer language such as, for example, Java, C++ or Perl using, for example, conventional or object-oriented techniques. The software code may be stored as a series of instructions, or commands on a computer readable medium, such as a random access memory (RAM), a read only memory (ROM), a magnetic medium such as a hard-drive or a floppy disk, or an optical medium such as a CD-ROM. Any such computer readable medium may reside on or within a single computational apparatus may be present on or within different computational apparatuses. For example, code for the forms engine 84(c) could reside on a server, client, or both a server and a client. A computer readable medium comprising code for a forms engine may encompass each of these possibilities.

Referring to FIG. 3(b), XML data from the data connect element 36(a) shown in FIG. 3(a) may be input into the decryption module 70 at data central server 44. The decryption module 70 decrypts the XML data and forwards it to a data queue 72. Data is then sent from the data queue to the site specific data filter 74 where the data sets are filtered according to their originating sites. Then, the normalizing engine 76 normalizes the data into a standard format. The normalizing engine 76 takes the site specific XML data and maps it to a standard data format using a dictionary database 82. The standardized data is then filtered using the scenario 78, and the scenario 78 is used to identify potential candidates. After the scenario 78 screens the patient information, the study specific converter 80 converts the patient information for the potential candidates into site study specific information 80 before it is sent to the client computers operated by the study coordinator and/or investigator.

The data central server 44 may also perform other functions. For example, the data central server 44 may track patient information by patient in addition to tracking information by site. This would be helpful in instances where the patient might use two or more healthcare institutions (e.g., a doctor, a dentist, and a hospital) and data pertinent to that patient is desirably collected and tracked.

I. Patient Recruitment

As noted in the Background of the Invention section above, because of the delays associated with conventional patient recruitment processes, many candidates can be lost using conventional recruitment processes. Illustratively, a study may relate to studying the various levels of blood components in a patient's blood during a heart attack. The blood sample needed for this study may only be good at the time of the heart attack. Using traditional recruitment methods, a patient with the heart attack may not be readily identified by an investigator. By the time the patient is identified as one that is a suitable candidate, the patient's sample may no longer be useful and/or it may be difficult to obtain the patient's consent because the patient has left the hospital.

Using embodiments of the invention, however, this patient can be identified to a study coordinator and/or investigator in substantially real time, and the study coordinator and/or investigator can get that patient's consent substantially contemporaneously with the actual medical event. For example, with 1000 patients, a typical scenario run can take as little as 0.8 seconds to run. The throughput using embodiments of the invention can be on the order of 500 messages processed per minute. Samples can also be taken while the patient is still at the hospital and testing on that sample can be done in a timely manner.

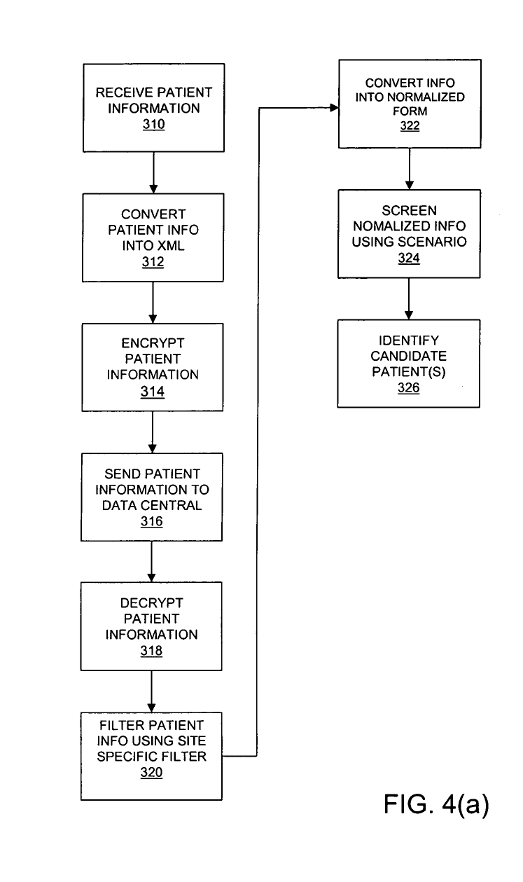

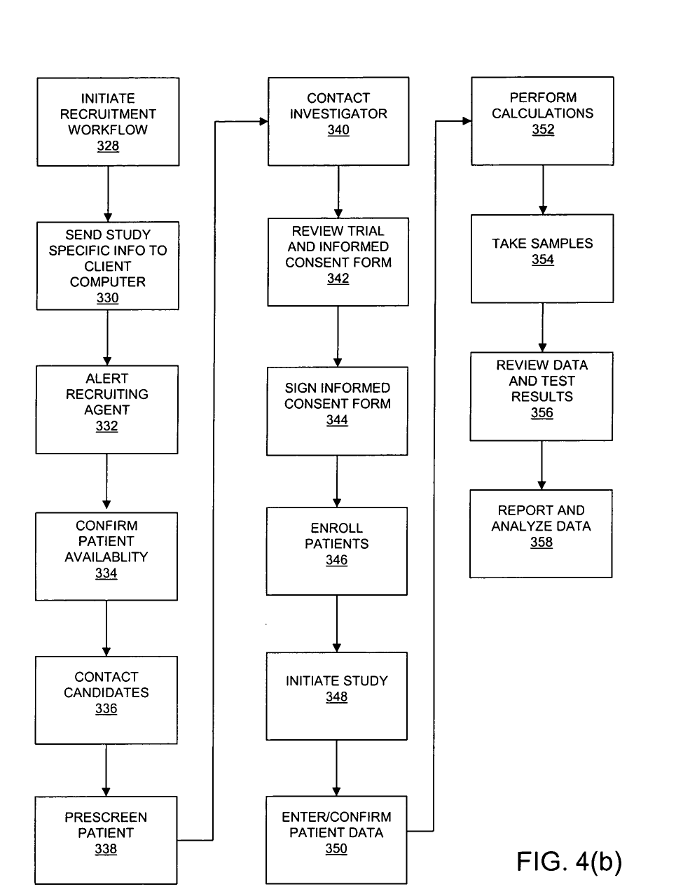

FIG. 4(a) shows a flowchart for a recruitment process according to an embodiment of the invention. FIG. 4(b) shows a flowchart for a workflow process according to an embodiment of the invention. The illustrated methods can be described also with reference to FIGS. 2 and 3(a)-3(b). It is understood that embodiments of the invention are not limited to the particular order of steps shown and embodiments of the invention need not include all such steps and/or may include additional steps. Also, any of the steps shown in FIGS. 4(a)-4(b) can be combined in any suitable manner without departing from the scope of the invention.

Referring to FIG. 4(a), patient information is received at the data connect elements 36(a)-36(c) through multiple data feeds 34(a)-34(c) at or associated with various sites 32(a)-32(c) (step 310). As noted above, in some embodiments, the data feeds 34(a)-34(c) can provide HL7 data or some other type of data to the data connect elements 36(a)-36(c). The received patient information includes site specific patient data sets from sources such as pharmacies, doctors' offices, laboratories, etc. The patient information may be from patient records at the various sites 32(a)-32(c). In some instances, the patients may have previously given their consent for screening for a possible future study.

Data conversion modules 66 in the data connect elements 34(a)-34(c) then convert the patient information including the site specific data sets from an HL7 data format into an XML data format (step 312), or some other suitable data format. This is done so that the patient information can be sent to the data central server 44 over an external network such as the Internet. Presently, XML data is generally more compatible with external networks than HL7. If desired, identifiers (e.g., identification numbers) which identify the data connect elements 34(a)-34(c) or the sites 32(a)-32(c) may be assigned to the site specific data sets by the data connect elements 34(a)-34(c).

In other embodiments, the patient information including the patient data sets need not be transformed at the data connect elements 36(a)-36(c). For example, in other embodiments, the patient information need not be transformed at all if the patient information from the sources is in a common format and uses common terms. In another alternative, patient information could be transformed (if needed) at the data central server 44 instead of at the data connect elements 36(a)-36(c).

After converting the patient information including the patient data sets into XML, an encryption module encrypts the converted patient information (step 314) so that it can be transmitted through the external communication medium 46(a) to the data central server 44. Encryption methods and software are known to those of ordinary skill in the art. The converted patient information is confidential and private and is securely transmitted through the Internet. The processing of the patient information makes it compliant with HIPAA or other rules or regulations.

The encrypted patient information is sent through the firewalls 38(a)-38(c) at the various sites 32(a)-32(c) to the data central server 44 through the communication medium 46(a) (step 316). The encrypted patient information that is sent from the data connect elements 34(a)-34(c) may be in the form of SOAP messages. The SOAP messages may be transported over the communication medium 46(a) using any suitable Internet protocol including SMTP, MIME, HTTP, HTTPS, etc.

At the data central server 316, the patient information is decrypted (step 316) by the decryption module 70. After the patient information including the patient data sets is decrypted, it then passes to a data queue 72 where it waits to be processed by an optional site specific data filter 74 (step 320). The patient information in the queue 72 can include a number of patient information messages from a variety of sites. The messages are received at a variety of different times. The site specific data filter 74 filters the messages by site so that the appropriate patient information and data sets can be further processed. By filtering messages by site, the data central server can then identify the standard data sets that correspond to the site specific data sets from that particular site.

After the patient information including the patient data sets is filtered with the site specific data filter 74, a normalizing engine 76 converts the patient information to a standard form (step 322). For example, the normalizing engine 76 converts site specific terms into standardized terms and converts site specific patient values into standardized patient values. This is done so that the patient information including the patient data sets can be filtered using the scenario 78.

In embodiments of the invention, the scenario and the different sites can use different terms, different patient values, and different units for the different patient values. For example, a term in a scenario may be “body temperature”. Patient information from a site may include the data set “temperature=99 degrees Celsius”. In this example, the site specific term for “body temperature” may be “temperature” and the site specific patient value associated with the site specific term would be “99”. The site specific unit associated with the site specific patient value “99” would be “Celsius”. Different sites may use site specific terms other than “body temperature” and site specific units other than “Celsius”. This makes it difficult to find patients that might satisfy a particular scenario for a study since the scenario also uses specific terms, specific values, and specific units.

The normalizing engine 76 and the dictionary database 82 solve this problem. The normalizing engine 76 changes the patient information into a standard form so that the scenario can filter all patient information, regardless of where it comes from. The dictionary database 82 provides data tables or other mapping elements that map site specific terms and units to standard terms and units. The dictionary database 82 also provides mapping from standard terms and units to study specific terms and units. Conversion factors for converting site specific units to standard units, and standard units to study specific units can also be present in the dictionary database 82.

Illustratively, one study specific term that is used by an investigator in a scenario may be “critical sodium”. This term may relate to the sodium concentration in a patient's blood sample at a predetermined time. A standard term associated with the study specific term may be “Na” and a standard unit for this standard term may be milliequivalents per liter (Meq/L). As shown in Table 1 below, the different sites, Site 1, Site 2, and Site 3 may have different ways of describing the concentration of sodium in a patient's blood and may use different units. For example, Site 1 may describe the study specific term “critical sodium” as “Na⁺ level” and may use the site specific unit “millimoles per liter”. Site 2 may describe the study specific term “critical sodium” as “sodium value” and may use the site specific unit “milligrams per liter”. Site 3 may describe the study specific term “critical sodium” as “sodium level” and may use the site specific unit “grams per liter”.

TABLE 1

| Site 1 | | Site 2 | | Site 3 | | | |
|---|---|---|---|---|---|---|---|
| Site | | Site | | Site | | Standard | |
| specific | | specific | | specific | | Standard | |
| term | Units | term | Units | term | Units | term | Units |
| Na⁺ | Mmoles/L | Sodium | Mg/L | Sodium | g/L | Na | Meq/ |
| level | | Value | | Level | | | L |

Because study investigators and the sites providing the patient information use different terms and units, it is difficult to determine if patients at the sites satisfy the predetermined critical sodium range defined in the scenario. For example, as shown below in Table 2, there can be three potential candidate patients at Sites 1, 2, and 3, respectively. Patient 1 at Site 1 may have a Na⁺ level of 137 millimoles per liter. Patient 2 at Site 2 may have a sodium value 2530 milligrams per mole. Patient 3 at Site 3 may have a sodium level of 3.910 kilograms per liter.

Embodiments of the invention address the problem of managing patient information from different sites having different site specific formats. In embodiments of the invention, patient information is received at the data central server 44, and the normalizing engine 76 converts each of the site specific terms and units into standard terms and units. Site specific patient values are also converted to the standard patient values so that all patient information is normalized to the standard. In this example, as shown below, after normalization, Patient 1 has a critical sodium value of 137 mEq/L, Patient 2 has a critical sodium value of 110 mEq/L, and Patient 3 has a critical sodium value of 170 mEq/L. Since all data sets are normalized to a standard, a scenario may be run against the standardized data sets to find suitable candidate patients.

TABLE 2

| | Patient 1 at Site 1 | Patient 2 at Site 2 | Patient 3 at Site 3 |
|---|---|---|---|
| Before | Na⁺ level = 137 | Sodium value = | Sodium level = |
| conversion | millimoles per liter | 2530 milligrams | 3.910 grams |
| | | per mole | per liter |
| After | Na = 137 mEq/L | Na = 110 mEq/L | Na = 170 mEq/L |
| conversion | | | |

The mapping between the site specific terms and site specific units to the standard terms and standard units, and any conversion factors that may be needed to convert the site specific patient values to standard patient values may be stored in the dictionary database 82 and may be used by the normalizing engine 76.

Once patient information from different sites is normalized to a standard (e.g., at the data central server 44), the normalized information is then screened using the scenario 78. More specifically, a subset of the standard data sets, or candidate data sets, is selected using the scenario 78. Candidate patients are then identified using the candidate data sets (which have standard terms and units) and are selected at the data central server 44 (step 326). In other embodiments, the selection of candidates could occur elsewhere (e.g., at a client computer). Since all patient information from incoming sites uses the same terminology and units, the patient information from the different sites can be screened together using one scenario. The information associated with the identified candidates may be further converted to study specific patient information, or may be directly sent to the study investigator or the study or site coordinator without further processing.

The standardized, site specific, or study specific patient information for selected and identified candidates may be stored in a separate data store (not shown) at the data central server 44 or at a location separate from it. Transport from the data central server to the data store may use Oracle SQL*Net or any other suitable software product.

The scenario 78 may run frequently (e.g., once every one or two minutes) and may filter for patient information that satisfies the scenario. The scenario 78 may be executed to filter data at regular pre-defined intervals, after receipt of notification of a new data set or a number of data sets, after notification of new patient data from a known patient or doctor, etc.

The scenario 78 may be set up in advance by an investigator and may be define a desirable population of patients. As noted above, an exemplary scenario for a study relating to the effects of smoking on sodium and potassium levels in people might be:
Critical Potassium (K<2.5 or K>6)
Critical Sodium (Na<120 or Na>160)
Female Smoker
Age (>35)
   Continuing with the previously described illustration, in this example, Patient 1 at site 1 would satisfy the “Critical Sodium” study specific term for this scenario while Patients 2 and 3 would not satisfy it. Patients 2 and 3 would therefore be excluded as potential study candidates while Patient 1 may be selected as a potential candidate if Patient 1 satisfies the other parameters of the scenario.

As illustrated above, embodiments of the invention allow for the rapid identification of potential study candidates from different sites. The identification can occur rapidly and accurately. Once suitable study candidates can be identified by the scenario 78, the workflow process can begin for the potential study candidates.

II. Patient Workflow

Referring to FIG. 4(b), once a list of suitable candidate patients is identified using the scenario 78, a recruitment workflow process is initiated by the workflow engine 84(b) (step 328).

The selected standard patient information including the candidate data sets is converted into study specific information using the study specific converter 80. Appropriate data conversions may also occur so that the candidate data sets are presented to the investigator or the study coordinator in a study specific manner. Table 3 below shows how additional mapping can occur between the standard terms and units, and study specific terms and units.

TABLE 3

| | |
|---|---|
| Standard | Study Specific |

| Standard Term | Units | Study Specific Term | Units |
|---|---|---|---|
| Na | Meq/L | Critical Sodium | Milligrams/L |

Once it is created, the study specific information including the study specific data sets is then sent from data central server 44 to one or more client computers 50, 52 (step 330). Each study specific data set may include a study specific term, an associated study specific value, and an optionally associated study specific unit.

The study specific information may be sent to the client computers 50, 52 in substantially real time. For example, the time needed to transfer patient information from site 1 32(a) for a patient, identify that patient as a potentially suitable study participant, and send study specific information about that patient to client computer 52 may take less than 10, 5, or 1 minute in embodiments of the invention. Preferably, the client computers 50, 52 are wireless devices so that the persons operating them can receive the patient information at essentially any location and at any time.

The study coordinator, investigator, or other user may operate client computer 52 and may thereafter be alerted that, for example, Patient 1 from site 1 satisfies the investigator's scenario (step 332). Appropriate information about Patient 1 may be sent to the client computer 52. For example, study specific patient values corresponding to the terms in the scenario may be sent to the client computer 52 where it can be viewed.

The study coordinator then contacts the investigator (typically a physician) to confirm patient eligibility (step 334). Alternatively, the investigator may be directly notified (without an intermediary study coordinator) that Patient 1 from site 1 satisfies the current scenario. The study coordinator may remove one or more patients from the identified candidate list if they are not deemed eligible and may provide appropriate comments as to why they have been removed.

In embodiments of the invention, the system identifies and alerts the appropriate recruiting agent. An alert can be delivered via an appropriate pre-configured device. For example, an e-mail status message with an active hyperlink can be sent directly to the investigator's e-mail account. The hyperlink can be activated to take the investigator into a browser, and may prompt the investigator for a username and password. The hyperlink may further direct the investigator to the specific alert within an alert view on an initial screen. Each alert type within the system can have a unique workflow to govern the actions that are to be taken to resolve the alert. This can prevent the investigator from being “over-notified” for a particular alert (e.g., the investigator only needs to be paged and/or e-mailed once per alert).

Then, the selected patient (or patients) is contacted by the study coordinator (or the investigator) (step 336), and/or a site coordinator. The patient is informed of the study and is asked if he is willing to participate in the study. If the patient is not willing to participate in the study, then the process may stop for the non-participating patient. Processed patient information for the non-participating patient may then be deleted from the system and/or the patient may be “de-identified” from the system. In a “de-identification” process, patient information is removed from the system so that it as if the patient information was never there. If the patient is willing to participate, then the process proceeds to step 338.

Then, the candidate patient (or patients) is prescreened (step 338). The patient may be interviewed and/or further data may be obtained from the candidate patient to determine if he is a suitable study candidate. Once the investigator determines that the candidate patient is a suitable candidate, the investigator contacts the study coordinator and confirms that the patient is an acceptable candidate for the study (step 340).

The consent of the identified patients is obtained. This involves a number of steps. First, the candidate patient is informed of the study and is presented with a consent form (step 342). Second, the candidate patient may be asked to sign the consent form (step 344). In some embodiments, the signed consent form may be on a tablet PC or other device which allows electronic signature capture. In this way, proof of actual consent may be obtained quickly and may be stored in an appropriate database.

Once the patient has consented, the patient and any other suitable patients are enrolled in the study (step 346). Once enrolled, the study is initiated (step 348). Study visits and activities may be scheduled for the consenting patients by the site and/or study coordinators. Data may then be collected from the patient.

Patient data is then entered and confirmed (step 350). The study coordinator has the ability to view the list of candidates. The study or site coordinator has the ability to retrieve forms via a wireless tablet PC (via standard browser forms) or other device to complete the data entry process for a patient visit. The patient data entry form uses available data to speed the data entry process (via an assisted data entry function which is described in further detail below). The patient data entry form can be a multi-page form (similar to a paper case report form) and includes data entry forms for all visits, along with fields for lab data and calculations. The recruiting agent has the ability to advance the patient after patient has been prescreened, or can remove the patient from the queue with appropriate reasons or comments.

If needed, calculations may then be performed (step 352) according to the study. For example, an investigator may take the collected patient data and may perform statistical calculations to prove a particular hypothesis. Calculations can be configured within the data entry forms—calculations are run in real-time based on available data and form rules (forms are customized by programmer/consultant).

If needed, samples may be taken from the enrolled patients (step 354). Sampling procedures may be coded into custom data entry forms. Test results are then reviewed (step 356), and the data is then analyzed and reported (step 358). Lab data values can be retrieved through the interface and entered into the patient data entry form by the study coordinator or site representative with the aid of assisted-data-entry functions.

In the methods described above, fees may be charged at any point in the methods. For example, a fee may be charged for each scenario that is run, and/or if a candidate match is found. Fees may also be charged for the candidate data sets that are produced by the data central server. In other embodiments, investigators or other users may be charged a subscription fee based on time used or based on a predetermined time span. In yet other embodiments, a graduated fee structure may be used so that patients that match the scenario better produce a higher fee than those that do not match the scenario as well. Other ways of charging fees are also possible in embodiments of the invention. Any combination of the above fee schemes may be used as well.

III. Exemplary Screenshots

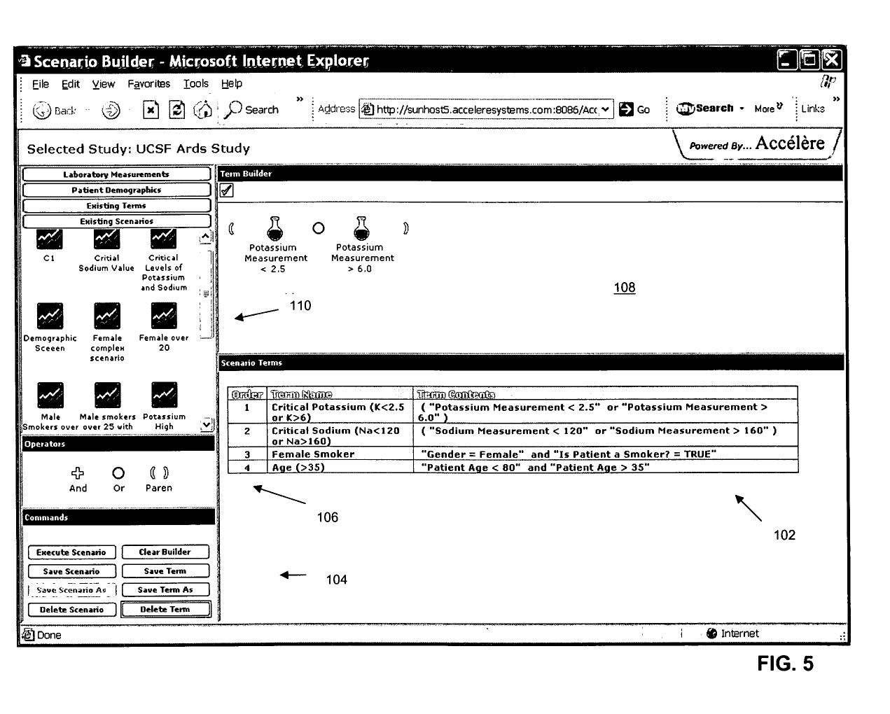

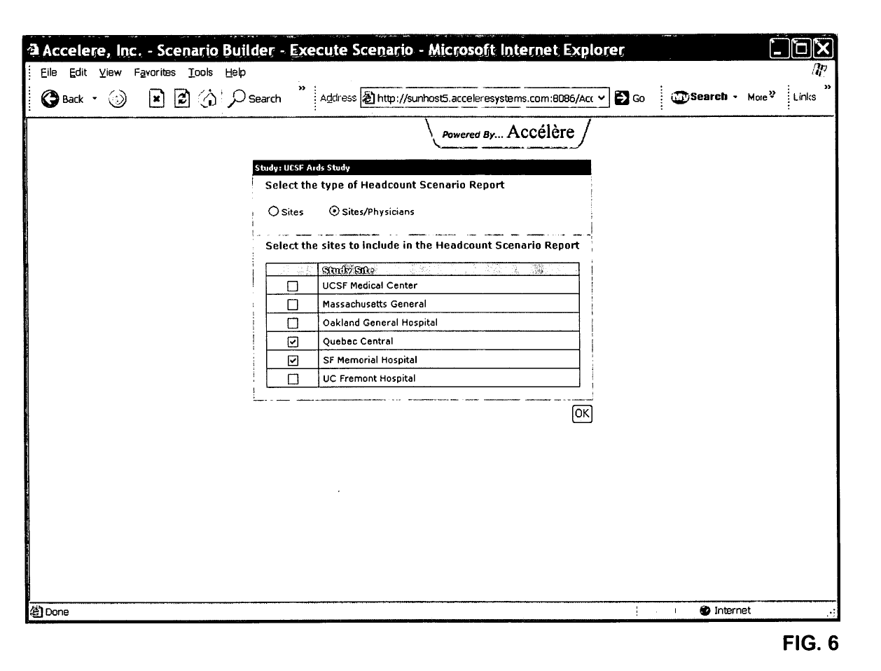

FIG. 5 shows a screenshot of a graphical user interface that can be used to create a scenario. As shown, there is a window 108 that shows a study term created by an investigator. In this example, a potassium measurement of less than 2.5 or greater than 6.0 will be one term that can be used to define a scenario. Window 102 shows the details of a scenario using the potassium measurement study term. Window 106 shows a number of icons that represent Boolean operators that can be used to create parameters for the scenario. Window 104 shows a number of buttons that allow the system to perform different functions including saving, deleting, and executing the created scenario, and deleting and saving terms in the scenario. Window 110 shows icons corresponding to difference scenarios that have been previously created. Previously created scenarios may be retrieved and modified so that data does not have to be re-entered.

FIG. 6 shows a screenshot of a graphical user interface which shows six different sites that can be searched for suitable candidates. As shown, the investigator can select which sites the scenario is to be run against. The investigator may also select a display that shows the sites from which suitable candidates come from and their physicians.

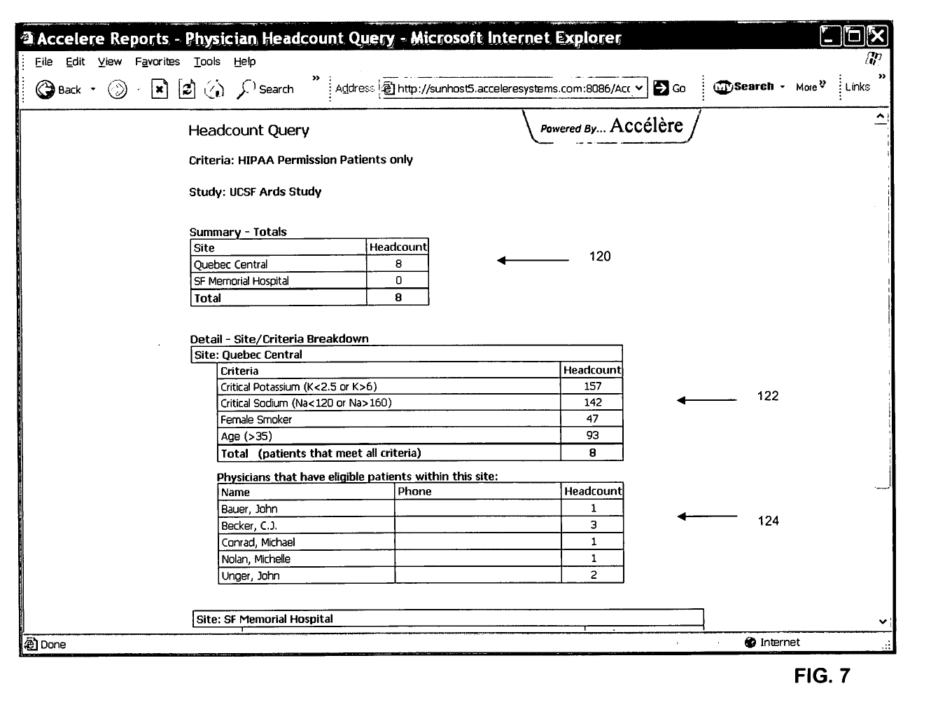

FIG. 7 shows a screenshot of a graphical user interface that shows results after the “OK” button is selected in FIG. 6. The screenshot shows a first table 120 showing the total number of patients that satisfy all of the criteria for the scenario at the different sites. The screenshot shows a second table 122 that shows how many patients satisfied each term in the scenario and the total number of patients meeting all terms of the scenario. The screenshot shows a third table 124 which shows the names of the physicians for those patients that have satisfied the scenario. Displaying the names of the physicians of the candidates is desirable, since they will eventually need to be contacted by the site or study coordinator.

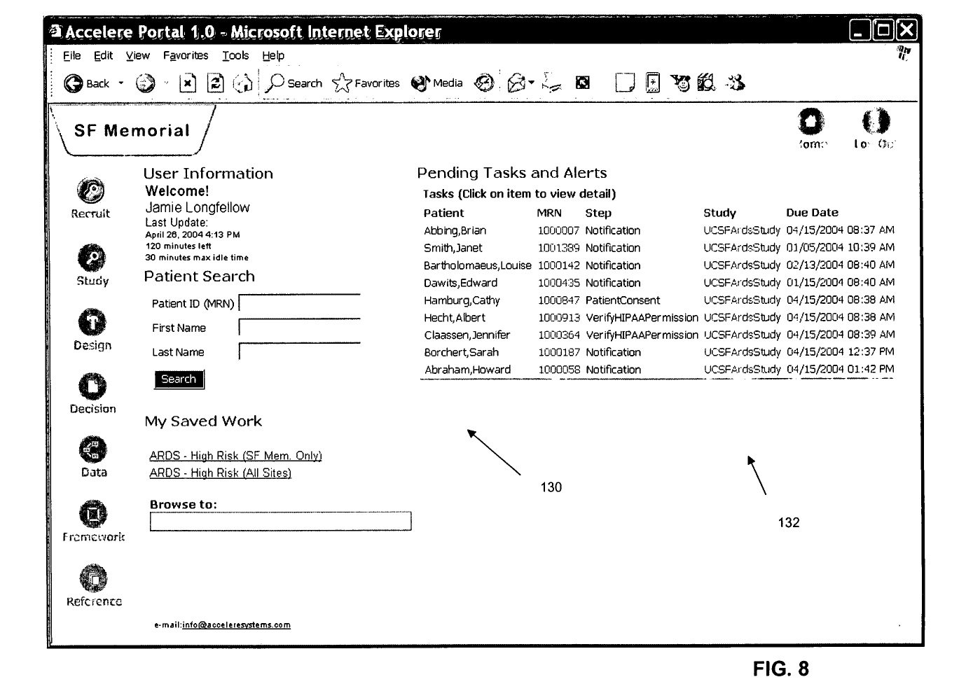

FIG. 8 shows a screenshot of a graphical user interface that is viewed by a study coordinator or an investigator. This screenshot shows that the workflow processes for many different patients can be run simultaneously, and that the different patients may be at different points in their respective workflow processes. Advantageously, a single screen can show a coordinator or investigator what to do on a particular day. The coordinator or investigator need not manually keep track of where each patient is in his or her particular workflow. Workflow tracking is advantageously done automatically in embodiments of the invention.

In the screenshot shown in FIG. 8, there is a list 132 of patient names, their medical record account numbers, the action items for those patients in the process, the names of the studies associated with the patients, and the due dates for action items. As shown, a number of patients have “notification” next to their names. This is a notification to the study coordinator or the investigator that suitable patients have been identified. A number of other patients also have “VerifyHIPAAPermission” next to their names. Here, the study coordinator and the investigator need to verify HIPAA permission with the patients. Last, one patient has “patient consent” next to her name. Here, the study coordinator needs to obtain the consent of the patient to move forward.

FIG. 8 also shows patient search windows 130 which allow the investigator or the study coordinator to search for patient information for a specific patient. If the investigator or coordinator wants to know more about a particular patient that is displayed, the investigator or coordinator may input a medical record number and/or name into the appropriate data fields.

As illustrated by FIG. 8, embodiments of the invention control the workflow for investigators and study coordinators on the floor. Embodiments of the invention allow investigators to conduct many simultaneous studies using across a common patient population using common resources. For example, it is possible to conduct two or three observational studies in a pulmonary medicine group at a hospital, and maintain over fifteen concurrent observational studies using the same resources. This can be done using custom workflows for each study, and by providing a common, role or person specific task schedule/list across workflow instances (e.g. patient A can be on Day 1 for study X, day 7 for study Y, and being screened for study Z, etc.). This specific example uses 3 separate workflows that are pre-defined (one per study), and one workflow instance per study for this patient. Thus, as illustrated above, embodiments of the invention collect workflow instances of different patients and put them into a common schedule for the coordinator on the floor.

When multiple scenarios are being run by different investigators across the same patient population, a single patient may be a suitable candidate for multiple studies. To address this problem, a quantitative ranking system can be provided to help identify the best study for the patient. This might be done by determining which scenario provides the best match (e.g., in term of percentage study parameters satisfied) for the patient. A weighted average process could also be used. For example, some study parameters may be more important than others and may be of greater weight then other parameters. Patients satisfying the more important study parameters would be a better match than patients that do not satisfy the more important study parameters.

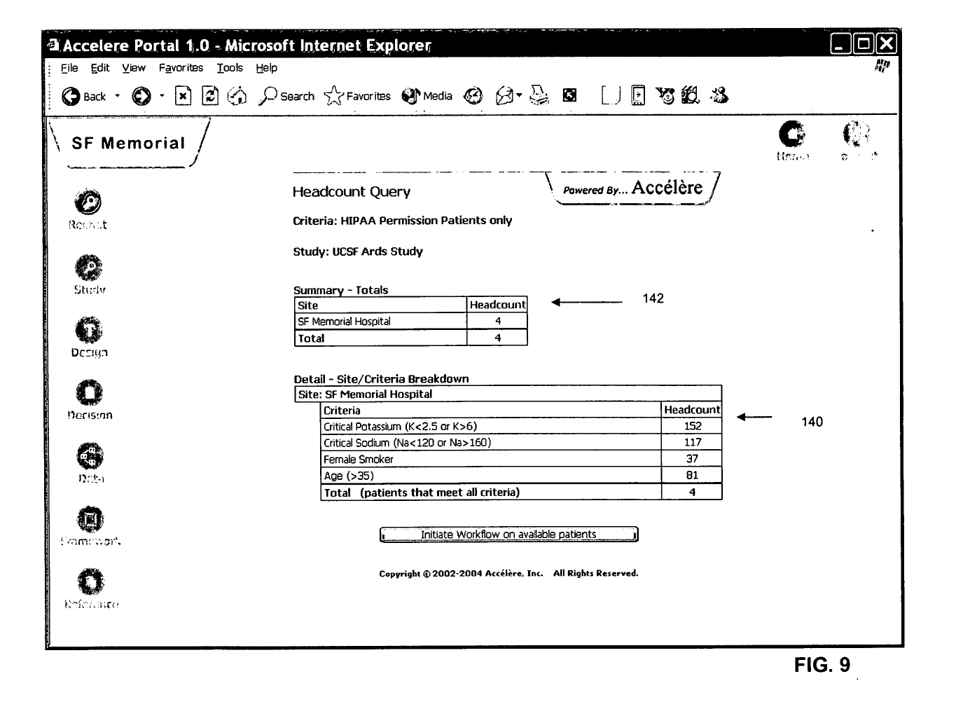

FIG. 9 shows a screenshot of a graphical user interface that shows the number of patients meeting each element of a scenario. Table 142 shows the total number of patients who have granted permission under HIPAA to participate in the study and who satisfy the scenario for the study. Table 140 shows a breakdown of the number of patients satisfying each term in the scenario. At the bottom of the screenshot, there is a button to “initiate workflow” on all patients.

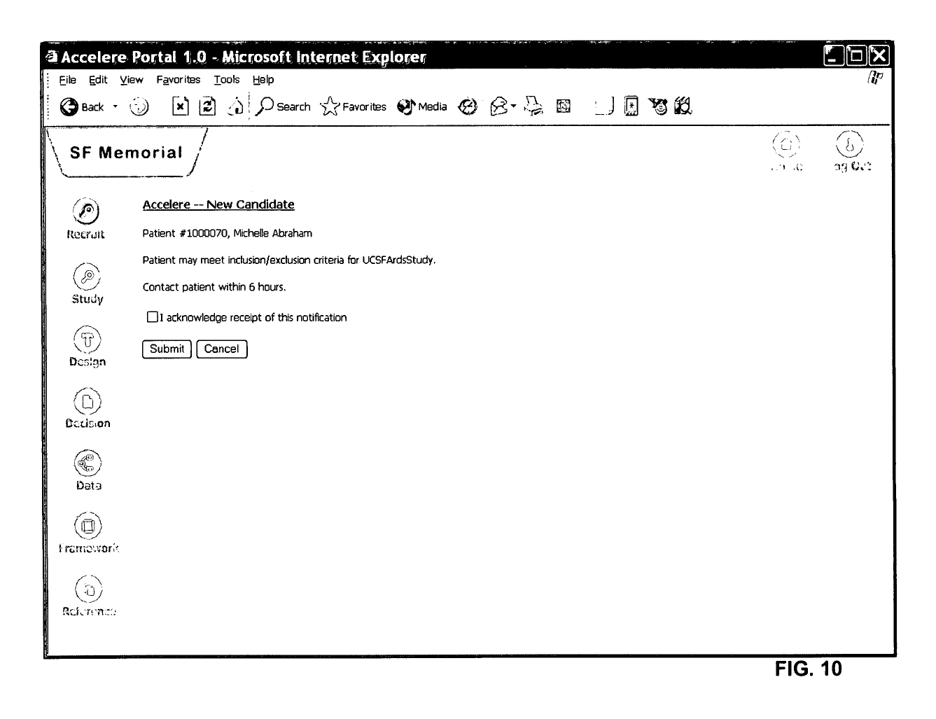

FIG. 10 shows a screenshot of a graphical user interface that can be used to notify a coordinator that a patient satisfying the scenario has been found. There is a note for the study coordinator to contact the patient within 6 hours. A box may be provided for the viewer to check to acknowledge receipt of the notification.

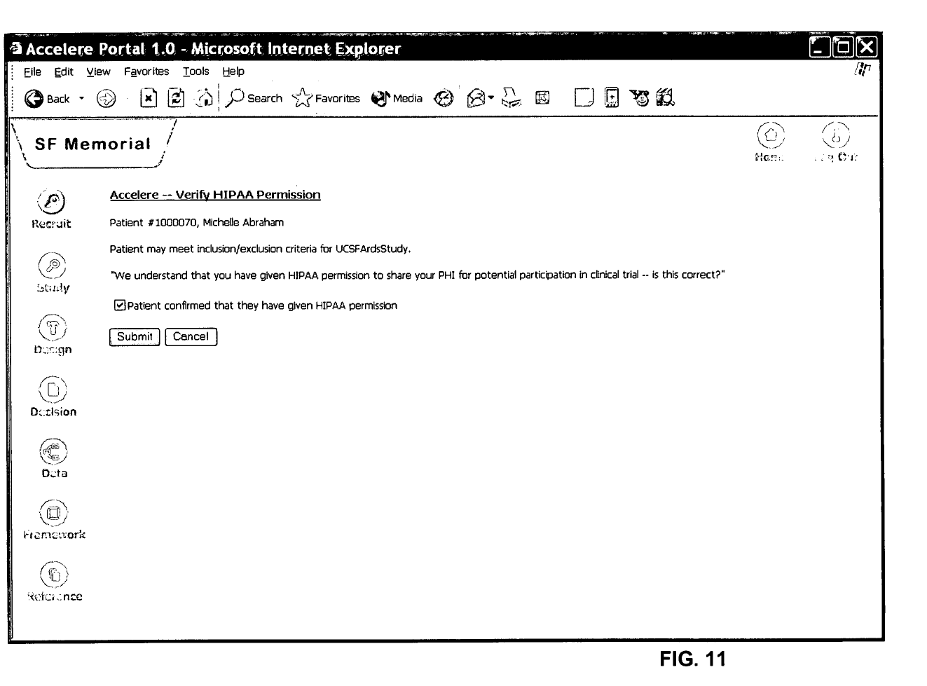

FIG. 11 shows a screenshot of a graphical user interface that shows that a patient has provided her consent. Here, the study coordinator selects a box which indicates that the patient has given HIPAA permission.

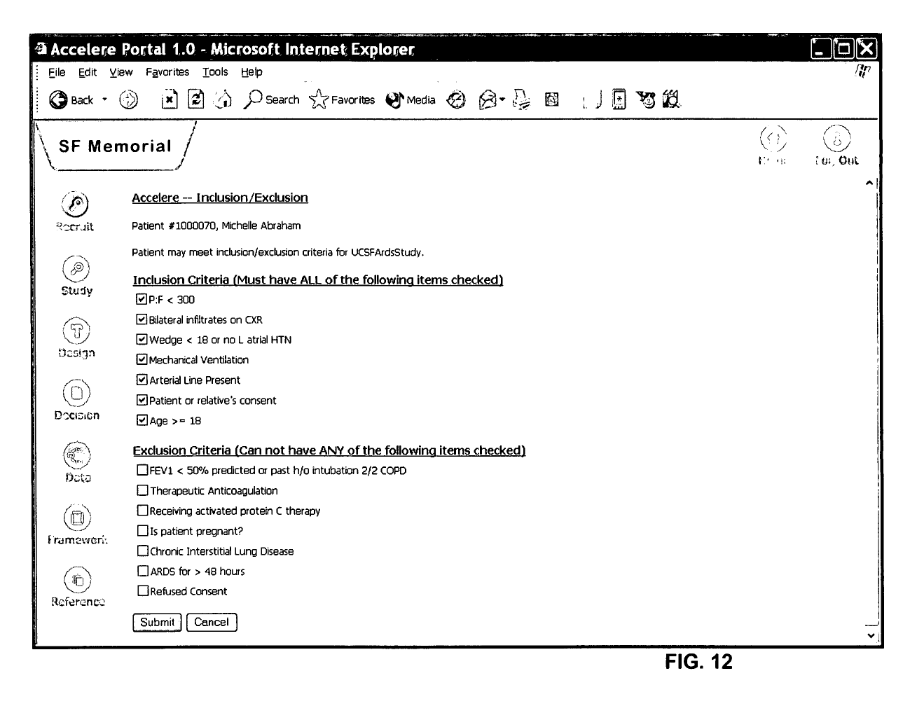

FIG. 12 shows a screenshot of a graphic user interface that list inclusion and exclusion criteria for a particular patient. The screenshot shows the name of a patient, the patient's medical record number, inclusion criteria for the study, and exclusion criteria for the study.

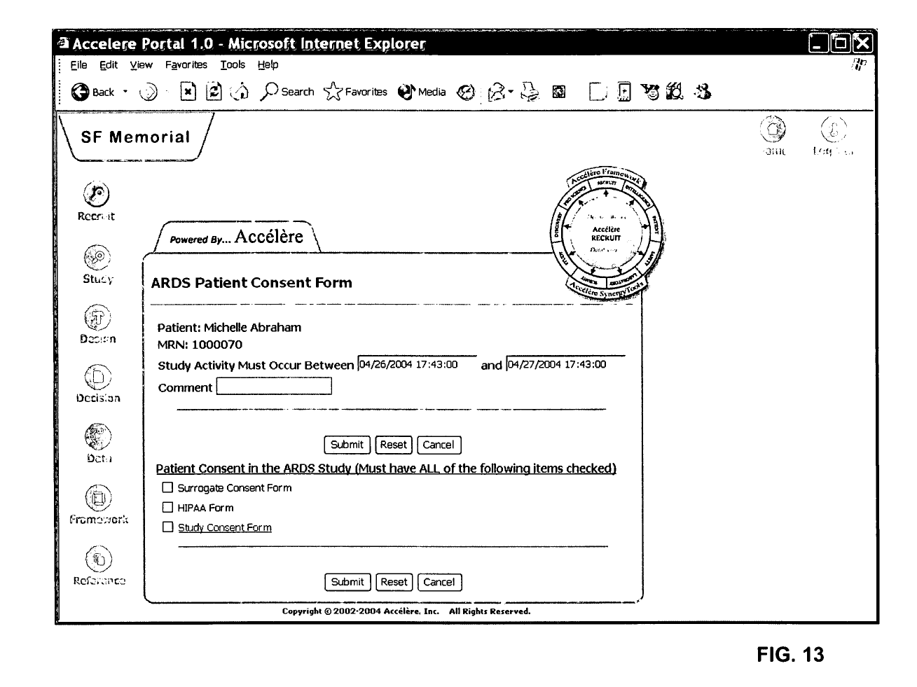

FIG. 13 shows a screenshot of a graphical user interface showing a patient consent form. The screenshot shows a patient consent form and is used by a study coordinator to verify that each consent form has been addressed by the study coordinator.

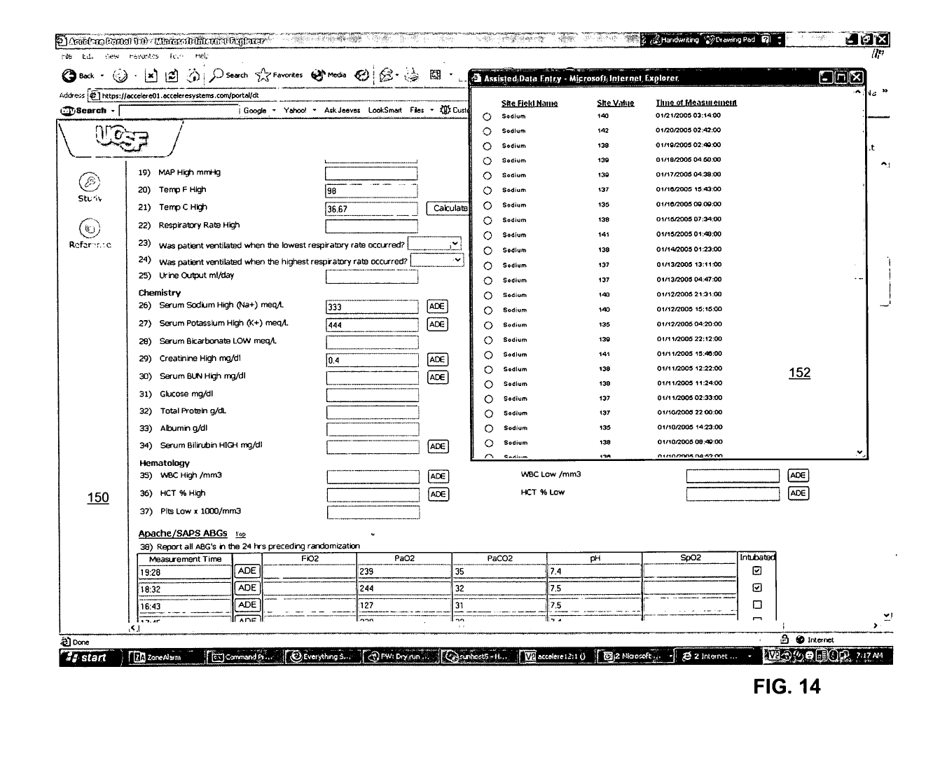

FIG. 14 shows a screenshot of a graphical user interface including an assisted data entry screen. Window 152 shows a window including sodium values corresponding to multiple measurement dates and times. Only those sodium values that need to be viewed by the investigator are displayed. For example, if some sodium values were taken long ago and are not particularly pertinent to the current study, then those values are not displayed to the investigator. This limits the dissemination of unnecessary information and helps protect patient privacy. This provides “assisted” data entry, since the investigator is automatically presented with a list of patient values and the investigator can pick any desired values for his study.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed.

Moreover, one or more features of one or more embodiments of the invention may be combined with one or more features of other embodiments of the invention without departing from the scope of the invention.

A recitation of “a”, “an” or “the” is intended to mean “one or more” unless specifically indicated to the contrary.

All patents, patent applications, publications, and descriptions mentioned above are herein incorporated by reference in their entirety for all purposes. None is admitted to be prior art.

1. A method comprising:
receiving site specific patient information including a plurality of site specific data sets for a plurality of patients from different sites at a server computer, wherein the site specific patient information was previously converted from a first data format to a second data format; and
converting the site specific patient information including the site specific data sets into standard patient information including standard data sets at the server computer.

2. The method of claim 1 further comprising:
comparing the standard data sets to a scenario to select a subset of data sets.

3. The method of claim 1 further comprising converting the standard patient information including the standard data sets into study specific information including study specific data sets.

4. The method of claim 1 wherein the first format is an HL7 format and the second format is a markup language format.

5. The method of claim 1 further comprising:
performing steps in a workflow process after converting the site specific patient information.

6. A system comprising:
a data connect element adapted to convert site specific patient information for a plurality of patients from a first format to a second format; and
a server computer adapted to convert the site specific patient information to standard patient information.

7. The system of claim 6 further comprising the one or more client computers operatively coupled to the server computer.

8. The system of claim 6 further comprising a plurality of data connect elements, wherein each data connect element is adapted to convert site specific patient information from the first format to the second format, and wherein the data connect elements are at different healthcare sites.

9. The system of claim 6 wherein the server computer comprises a workflow engine, wherein the workflow engine is adapted to execute a process to obtain consent to participate in a clinical or observational study from one or more patients selected from the plurality of patients.

10. The system of claim 6 wherein the server computer comprises code for assisted data entry of screened patient information.

11. The system of claim 6 wherein the server computer further comprises a normalizing engine that is adapted to normalize the site specific patient information.

12. A method of selecting a candidate for a study, comprising:
receiving a plurality of patient data sets at a data store located within an internal network, wherein each data set comprises at least one site specific term and an associated site specific patient value;
processing each of the received data sets as needed to transform the data sets into private data sets, and further, wherein the processed and private data sets are compatible with transport over an external network;
communicating the processed data sets over the external network to an external data store;
processing the communicated data sets as needed to transform each of the data sets into a standard data set including a canonical term and an associated canonical value;
comparing the transformed communicated data sets to a study scenario comprising a plurality of study parameters including the canonical data term and at least the associated canonical data value; and
processing the compared data sets to select a candidate data set for a study.

13. The method of claim 12 further comprising:
providing the selected candidate data set to a study coordinator.

14. The method of claim 12 further comprising associating the selected candidate data set with one or more individuals.

15. The method of claim 14 further comprising:
obtaining agreement of the associated individual to participate in the study.

16. The method of claim 12 further comprising:
charging a transaction fee for the selected candidate data set.

17. The method of claim 12 wherein, the plurality of candidate data sets further comprises a plurality of data sets from a single individual.

18. The method of claim 12 further comprising converting the candidate data set to a study specific data set.

19. The method of claim 12 wherein the processing of each of the received data sets as needed to transform the data sets into private data sets, and further, wherein the processed data sets are compatible with transport over an external network, further comprises:
assigning a client ID to the private data sets;
encrypting the private data sets; and
converting a format of the received data sets into a markup language transportable over the external network.

20. The method of claim 12 wherein the processing the communicated data sets as needed to transform each of the data sets into a standard data set including a canonical term and an associated canonical value comprises:
mapping the data sets to a list of a plurality of canonical data sets.
